(19) United States
(12) Patent Application Publication
(10) Pub. No.: US 2005/0282164 A1
(43) Pub. Date: Dec. 22, 2005

(54) Ultraphobic sample carrier having functional hydrophilic and/or oleophilic areas

(76) Inventors: Joachim Engelking, Neustadt (DE); Karsten Reihs, Koln (DE); Eckhard Nordhoff, Berlin (DE); Martin Muller, Berlin (DE)

Correspondence Address: PERMAN & GREEN, 425 POST ROAD, FAIRFIELD, CT, 06824, US

(21) Appl. No.: 10/505,617

(22) PCT Filed: Feb. 24, 2003
(86) PCT No.: PCT/EP03/01860

§ 371 Date: Aug. 11, 2005

(30) Foreign Application Priority Data
Feb. 22, 2002 (DE) 102 07 616.2
Nov. 26, 2002 (DE) 102 55 276.2

(51) Int. Cl. C12Q 1/68; C12M 1/34

(52) U.S. Cl. 435/6; 435/287.2

(57) ABSTRACT

The invention relates to a planar structure having an ultraphobic surface and at least one hydrophilic and/or oleophilic area that, in addition to being hydrophilic and/or oleophilic, has at least one additional functionality. The invention also relates to a method for producing the inventive planar structure and to the use thereof.

The present invention concerns a membrane with an ultraphobic surface and with at least one hydrophilic and/or oleophilic area which shows a further functionality in addition to the affinity for water and/or oil. Furthermore the present invention concerns a process for manufacturing the membrane according to the invention and its use.

In the area of medicinal chemistry and also in biological research and production these days, cumulative series tests must be carried out. In doing so, a large number of small, liquid samples, for example, are charged with different active ingredients in order to test their reaction to the respective active ingredient.

So-called microtiterplates or sample carriers are known from the current state of technology for such tests which, for example, show a multitude of regularly-spaced wells. Sample carriers are known from WO 98/45406 and DE 196128 928 whose surfaces are hydrophobic and are incorporated in the hydrophilic wells. A sample carrier with an hydrophobic surface is known from the German patent publication DE 197 54 978. Hydrophilic anchor areas are incorporated in these hydrophobic surfaces. However, state of the art sample carriers have the disadvantage that the hydrophilic areas can only be made relatively expensively, especially however, that the series tests with them must be performed only relatively expensively.

Therefore the purpose of the present invention is to make available a membrane which does not show the disadvantages of the state of the art.

This purpose is solved according to the invention with a membrane with an ultraphobic surface and with at least one hydrophilic and/or oleophilic area which shows at least one other functionality in addition to the affinity for water and oil.

It was extremely astonishing for and unexpected by the expert that the membrane according to the invention succeeds in significantly simplifying series tests and performing them with low particularly mechanical complexity.

A membrane for the purpose of the invention is any moulded padding with a surface of any shape. Preferably, however, the membrane is a plate with a level surface, especially preferred is a sample carrier which preferably however shows no recesses. The most preferred membrane according to the invention is a film showing an ultraphobic surface. Preferably the surface of the membrane according to the invention is basically planar; i.e. it shows the necessary topography for an ultraphobic surface, however no microvolumes in which liquid can gather

According to the invention, the membrane shows an ultraphobic surface. An ultraphobic surface for the purpose of the invention is distinguished in that the contact angle of a drop of water and/or oil that is on the surface is more than 150°, preferably more than 160°, and especially preferred more than 170° and the receding angle does not exceed 10°. Receding angle means the angle of inclination of a basically planar but structured surface from the horizontal, at which a stationary drop of water and/or oil with a volume of 10 μl is moved due to gravity for an inclination of the surface. Such ultraphobic surfaces are revealed, for example, in WO 98/23549, WO 96/04123, WO 96/21523, WO 99/10323, WO 00/39368, WO 00/39239, WO 00/39051, WO 00/38845 and WO 96/34697 which are hereby incoporated as references and thus count as part of the disclosure.

In a preferred embodiment, the ultraphobic surface shows a surface topography for which the local frequency of the individual Fourier components and whose amplitude a (f) expressed by the integral S (log(f))=a(f)*f calculated between the integration limits log(f₁/μm⁻¹)=−3 and log(f₂/μm ⁻¹)=3 is at least 0.3 and which consists of an hydrophobic or particularly oleophobic material or are coated with a durable hydrophobic and/or particularly durable oleophobic material. Such an ultraphobic surface is described in the international patent registration WO 00/39249, which is hereby incorporated as a reference and thus counts as part of the disclosure.

According to the invention, the membrane also shows hydrophilic and/or oleophilic areas. Hydrophilic and/or oleophilic areas for the purpose of the invention are areas on which a drop of water or oil can be deposited; i.e. a drop of water or oil, which is brought into contact with the hydrophilic and/or oleophilic area by a pipette system, remains there and detaches itself from the pipette system. Preferably, a drop of water or oil with a volume of 10 μl on the hydrophilic and or oleophilic areas have a contact angle <120°, preferably <110°, especially preferred <90° and/or the receding angle of this drop exceeds 10°. Furthermore, according to the invention, the hydrophilic and/or oleophilic areas show at least one other functionality in addition to the affinity for water and/or oil.

A further functionality for the purpose of the invention is any other chemical and/or physical characteristic which the material of the hydrophilic and/or oleophilic areas shows in addition to repelling water or oil and which an be used technically. For example, but not limited to these only, the properties are mentioned here: The areas can show at least one surface,
which makes a bond with other molecules;
which catalyses chemical reactions;
which releases at least one substance, which makes a bond with other molecules;
which has the effect of a reagent for samples to be tested;
which releases at least one substance, which has the effect of a reagent for samples to be tested;
which shows a different optical property (absorption, reflection, transmittance, remittance, luminescence, diffusion) than the environment;
which emits light when subjected to heat;
which shows a different heat transfer ability than the environment;
which shows a different acoustic property (e.g. sound absorption, acoustic velocity) than the environment;
which shows a different surface friction than the environment;
which shows a different absorption behaviour (e.g. absorption speed, equilibrium) than the environment;
which shows a different electrical property (e.g. conductability, capacitivity) than the environment;
which shows a different magnetic property (e.g. susceptibility) than the environment;
which shows a different surface diffusion behaviour than the environment;
which contains a radioactive substance;
which shows a different α, β or γ emission than the environment;
which shows a different specific surface than the environment;
which shows a different surface topography than the environment;
which shows a different porosity than the environment;
which shows a different surface coverage of an adsorbate than the environment;
which shows a different molecular weight of a polymer than the surrounding polymer surface;
which shows a different electrochemical potential than the environment;
which shows a different surface loading thickness than the environment;
which shows a different electrokinetic potential (zeta potential) than the environment;
which using one of the chemical or physical properties mentioned above releases at least one substance having the effect of a reagent for the samples to be tested;
which using one of the chemical or physical properties mentioned above releases at least one substance which bonds with other molecules and/or splits molecules e.g. biomolecules specifically or not specifically.

Preferably the hydrophilic and/or oleophilic areas are respectively completely enclosed by an ultraphobic area. Using this embodiment it is possible to anchor a drop of liquid metered on to the hydrophilic and/or oleophilic area there relatively firmly.

Also preferred is that the hydrophilic and/or oleophilic areas are arranged on the ultraphobic surface according to a very specific pattern. Thus, for example, a raster, a so-called array, can be produced in which the hydrophilic and/or oleophilic areas can then be easily moved towards for series tests by, for example, a machine.

The hydrophilic and/or oleophilic areas can show any shape and size. Preferably, however, they have an area from 1 μm²-10 mm². A drop of liquid with a diameter of preferably 5 nm-5 mm can be deposited on such an area and preferably anchored so that it does not detach itself hanging down from the membrane according to the invention. Preferably the hydrophilic and oleophilic areas have a minimum distance of 10 μm from each other.

The hydrophilic and/or oleophilic areas can be incorporated in any conventional way in the membrane or applied on the ultraphobic surface. Preferably, however, the hydrophilic and/or oleophilic area is respectively at least one deposit on the ultraphobic surface. This deposit can be liquid or solid. In the case of a liquid deposit, it must preferably at least at room temperature not be or be only slightly volatile. The deposited substance can be produced for example using a corresponding temperature of the ultraphobic surface or using substances which are preferably detached and/or suspended on the ultraphobic surface preferably in the form of drops, and for which the solvent or liquid phase is then evaporated. In doing so the ultraphobic surface must be wettable by the solvent or the liquid phase. Details for this can be found from the parallel registrations with the internal file numbers Sy 0028 and Sy 0029 lodged with the German patent and trademark authority which are hereby incorporated as references and thus count as part of the disclosure. The solid deposit can also be a thin film of a solid substance. The deposit can also be an adsorbate whose layer thickness is only a fraction of a monolayer or layers several molecules thick. The deposit can for example be detached again from the ultraphobic surface with the corresponding solvents so that the ultraphobic surface can be re-used.

Preferably the hydrophilic and/or oleophilic area shows the additional functionality of a MALDI matrix; i.e. the hydrophilic and/or oleophilic area is simultaneously a is MALDI matrix for performing the so-called MALDI mass spectrometry which is described for example in Nordhoffet al. “MALDI-MS as a new method for the analysis of nucleic acid (DNA (deoxyribonucleic acid) and RNA (ribonucleic acid) with molecular masses up to 150,000 Dalton, Application of modern mass spectrometric methods to plant science research”, Oxford University Press (1996) pages 86-101. This publication is hereby incorporated as a reference and thus counts as part of the disclosure. Preferred MALDI matrices are 3-hydroxypicolinic acid, α-cyano-4-hydroxycoumarin acid, 2.5 dibydroxybenzoic acid, sinapic acid, 2 4 6 trihydroxyacetophenone nitrobenzyl alcohol, nicotinic acid, ferulic acid, caffeic acid, 2-aminobenzoic acid, picolinic acid, 3-aminobenzoic acid, 2,3,4-trihydroxyacetophenone, 6-aza-2-thiothymine, urea, succinic acid, adipic acid, malonic acid or their mixture. These MALDI matrices are dissolved for example in acetonenitrile and preferably applied on the ultraphobic surface as drops of liquid and the solvent is then evaporated there so that the MALDI matrix is available preferably as a crystallised structure at certain points on the ultraphobic surface and thus shows the hydrophilic and/or oleophilic areas on which the samples to be analysed can be metered.

Preferably the membrane, which especially preferred is a sample carrier, shows many places each with a MALDI matrix, which are respectively completely enclosed by the ultraphobic surface. A sample carrier can show the same matrix or different matrices at these places.

The ultraphobic surface non-wetted samples to be analysed are usually metered as liquid on the preferably crystalline MALDI matrices and preferably apply a little solvent at least partly to them. While the solvent is evaporating again the MALDI matrix crystallises again and the sample molecules to be analysed are embedded in the MALDI matrix or bond to the surface of the MALDI matrix. The samples so prepared can then be analysed with a corresponding mass spectrometer.

This preferred embodiment of the present invention has the advantage that sample carriers can be made available on which the MALDI matrix or several MALDI matrices already exists at predefined positions. The user still has to only apply the samples to be analysed to the respective MALDI matrices so that the analysis is significantly simplified for him. He does not have to make and store the MALDI matrices and also does not have to keep any equipment with which the MALDI matrices can be applied to the sample carriers and dried. The MALDI matrix, which also represents the hydrophilic and/or oleophilic area according to the invention of the membrane, has the effect of an anchor for the sample liquid which does not wet the ultraphobic surface and therefore does not come into contact with this so that the sample cannot be contaminated by substances on the ultraphobic surface. This is especially very important for MALDI mass spectrometry because the sample liquid is evaporated and in doing so the existing contaminations have increased solvent concentration. Therefore high-quality mass spectrums can be reproducibly determined with the membranes according to the invention. The ultraphobic surfaces can be cleaned after each use and used again.

In another preferred embodiment of the present invention, the hydrophilic and/or oleophilic areas also still show the functionality of an affinity matrix; i.e. the hydrophilic and/or oleophilic areas are simultaneously also affinity matrices. An affinity matrix for the purpose of the invention only bonds certain molecules of a molecule mixture. The bonding can be reversible or irreversible. The bonded molecules can be separated for example by washing the mixture and then analysed. Preferably the selectively bonded molecules are biomolecules and/or biological material, particularly DNA, RNA (ribonucleic acid), nucleic acid, nucleic acid analoga, spiegelmers, aptamers, ribozymes, polypeptides, peptides and/or proteins. These biomolecules can be analysed for example by allowance of at least one MALDI matrix and MALDI mass spectrometry afterwards. The affinity matrix which simultaneously represents the hydrophilic and/or oleophilic area is preferably crystalline. Examples for an affinity matrix are made from the conventional solid phase chromatography chemical groups such as e.g.: anion exchange chromatography: —NH₂, —(CH₂)₄—NH₂ or (CH₂)₆—NH₂ or cation exchange chromatography; —C₆H₄—SO₃H or reversed phase chromatography: —(CH₂)₃—CH₃, —(CH₂)₇CH₃, —(CH₂)₁₇—CH₃. Especially preferred however is that the affinity matrix is simultaneously also still a MALDI matrix. Such substances are for example α-cyano-4-hydroxycoumarin acid, 2,4,6 trihydroxyacetophenone, caffeic acid, sinapic acid or their mixture. The affinity matrices are dissolved for example in acetone, acetone/acetic acid water mixture, acetonenitrile, ethanol, isopropanol or their mixture and preferably applied on the ultraphobic surface as drops of liquid and then the solvent is evaporated there so that the affinity matrices exist preferably as a crystalline structure at certain points on the ultraphobic surface and thus represent the hydrophilic and/or oleophilic areas on which the samples to be separated and then to be analysed can be metered.

A biomolecule for the purpose of the present invention is any molecule that any virus or organism with one or more cells is made from during its lifecycle. Biomolecules contain at least one oxygen, nitrogen, sulphur and/or phosphorous atom. For example, the following could be named as biomolecules: spiegelmers, aptamers, ribozymes, peptides, polypeptides, proteins, antibodies, nucleic acids, nucleic acid analogas, DNA, double-stranded DNA, RNA, double-stranded RNA/DNA, vitamins, carbohydrates, hormones, glycopeptides, glycoproteins, lipides, fatty acids and cholesterols.

Biological material for the purpose of the invention contains at least one biomolecule. In this connection it can also be a matter of large quantities of the same or different biomolecules. These can exist unorganised next to each other or form functional units due to change effects. Examples of these are complex proteins, genomes, cell nucleii, ribosomes, cells, united cell structures, membranes or complete organisms.

This preferred embodiment of the present invention has the advantage that sample carriers can be made available on which an affinity matrix or several affinity matrices already exist at defined positions. Different affinity matrices on a sample carrier have the advantage that different molecules can be selectively bonded respectively on each affinity matrix. The user only has to still apply the sample liquid to be separated on each affinity matrix so that the separation of individual bondings is significantly simplified for him. He does not have to make and store the affinity matrices and also does not have to keep any equipment with which the affinity matrices are applied to the sample carrier and dried. The affinity matrix, which also represents the hydrophilic and/or oleophilic area according to the invention of the membrane, has the effect of an anchor for the sample liquid which does not wet the ultraphobic surface and therefore does not come into contact with this so that bonded molecules on the affinity matrix cannot be contaminated by substances on the ultraphobic surface. The selectively bonded molecules on the affinity matrix are preferably biomolecules and/or biological materials, especially preferred are nucleic acids, nucleic acid analogas, spiegelmers, aptamers, ribozymes, polypeptides, peptides and/or proteins. The bonded molecules can be analysed afterwards or are available for further reactions. The ultraphobic surfaces can be cleaned after each use and used again.

In another preferred embodiment of the present invention, the hydrophilic and/or oleophilic area is at least one substrate on which at least one molecule, preferably a biomolecule and/or biological material, can be irreversibly or reversibly bonded covalently. Examples of such substrates are polyacrylamide, polyethyleneglycol, polyvinyl alcohol, agarose, nylon, nitrocellulose and/or methylcellulose. Preferably the substrate shows a three-dimensional preferably porous structure whose pores are preferably 1-100 nm, especially preferred 1-20 nm and very particularly preferred 1-10 nm. Other examples for such substrates are inorganic bonds. Any substances can be bonded to these substrates. Preferably, however, the substances are certain selected biomolecules and/or biological materials which are immobilised on the substrates and are then available for further biochemical or biological experiments or can be stored. In this way biomolecule chips also called biomolecule arrays for example can be made available on which preferably several different biomolecules and/or biological materials preferably however of the same genus are immobilised.

The biomolecule chips are also an object of the present invention. Preferred biomolecules are DNA, RNA, peptides and/or proteins. For example, so-called DNA or protein chips can be made with these biomolecules in which many different DNA or protein molecules are bonded in the form of a defined raster. The immobilised molecules on these chips are then incubated with a sample solution. In the case of DNA-DNA, DNA-RNA or RNA-RNA interactions this operation is also known as hybridisation. If proteins are immobilised, particularly protein-protein, protein-DNA, protein-RNA or the change effect of the immobilised proteins with pharmacological agents are interesting. It is essential to the invention that both the immobilisation of the molecules as well as their further use takes place on an ultraphobic surface so that a contamination of the molecules with substances stored on the ultraphobic surface is practically impossible and background signals are basically reduced during the analysis of the immobilised molecules, preferably biomolecules. The ultraphobic surfaces can be cleaned after each use and used again.

In another preferred embodiment of the present invention, the hydrophilic and/or oleophilic area is a biomolecule or biological material which is applied preferably dissolved and/or suspended to the ultraphobic surface and for which the solvent or the liquid phase is then evaporated. The solvent or the liquid phase must wet the ultraphobic surface. The biomolecules or biological materials immobilised in this way are then available for example for further biochemical or biological experiments. In this way, biomolecule chips also called biomolecule arrays for example can be made available on which preferably several different biomolecules preferably of the same genus are immobilised. The biomolecule chips are also an object of the present invention. Preferred biomolecules are DNA, RNA, peptides and/or proteins. For example, so-called DNA or protein chips can be made with these biomolecules in which many different DNA or protein molecules are bonded in the form of a defined raster. The immobilised molecules on these chips are then incubated with a sample solution. In the case of DNA-DNA, DNA-RNA or RNA-RNA interactions this operation is also known as hybridisation. If proteins are immobilised, particularly protein-protein, protein-DNA, protein-RNA or the change effect of the immobilised proteins with pharmacological agents are interesting. It is essential to the invention that both the immobilisation of the molecules as well as their further use takes place on an ultraphobic surface so that a contamination of the molecules with substances stored on the ultraphobic surface is practically impossible and background signals are basically reduced during the analysis. In this embodiment of the membrane according to the invention it is particularly advantageous that the biomolecules adhere directly to the ultraphobic surface without another substance so that the manufacture of these membranes is especially simple and economic. The ultraphobic surfaces can be cleaned after each use and used again.

The membranes according to the invention are suitable for the analysis of any kind of liquid such as those known for example from chemical research or biotechnology. The membrane according to the invention is particularly suitable for expression, mass spectrometric and/or optical analysis of biomolecules and/or biological materials especially nucleic acids, nucleic-acid analogas, spiegelmers, aptamers, ribozymes, polypeptides, proteins and/or peptides. These applications are also objects of the present invention.

An optical analysis for the purpose of the present inventions is described in the German parallel registration with the file number DE 102 07 614 lodged with the German patent and trademark authority. Details of mass spectrometric analysis, particularly the MALDI process, can be found in the parallel registration with the file number DE 10207615 also lodged with the German patent and trademark authority. Both registrations are hereby incorporated as references and thus count as part of the disclosure.

Furthermore, the hydrophilic and/or oleophilic area can also be a peptide nucleic acid which is hybridised with single-stranded DNA.

Another object of the present invention is a process for manufacturing the membrane according to the invention in which a functional substance on the respective ultraphobic surface which is dissolved and/or suspended in a solvent which wets the ultraphobic surface is preferably metered in drops and the liquid phase or the solvent is evaporated afterwards.

The process according to the invention is simple and economic to execute. The functional substance, which simultaneously represents the hydrophilic and/or oleophilic areas, can be detached again after each use and the ultraphobic surface used again.

Preferably several different substances are metered onto the ultraphobic surface so that so-called arrays can be produced. Preferably the functional substances are MALDI matrices, affinity matrices, biomolecules especially DNAs, proteins and/or bond molecules. DNA or protein chips can thus be manufactured with the process according to the invention.

Reference should be made to the above with respect to the application concerning MALDI matrices, affinity matrices, biomolecules and bond molecules.

In a preferred embodiment of both membranes according to the invention, the ultraphobic surface is designed as disposable.

A membrane with several layers with a first layer with an ultraphobic surface and a carrier, where the first layer is applied reversibly to the carrier and the maximum local flatness deviation of the membrane is 100 μm preferably <20 μm, is particularly suitable for this embodiment.

This membrane has the advantage that the first layer with the ultraphobic surface can be detached from the carrier after one or after several uses and can be replaced with a new first layer so that it is impossible that this first layer is contaminated by previous experiments. The first layer with the ultraphobic surface is particularly economic to make as disposable. As a result of the defined flatness according to the invention, it is guaranteed that the membrane can be used in all current mass spectrometers and/or optical analysis equipment.

In a preferred embodiment of the membrane the first layer is glued on to the carrier.

Furthermore, it is preferred there is an electrical contact between the first layer and the carrier. This embodiment is particularly advantageous for mass spectroscopic analyses.

The preferred membrane has diverse uses, however it is especially suitable for mass spectroscopic and/or optical analyses.

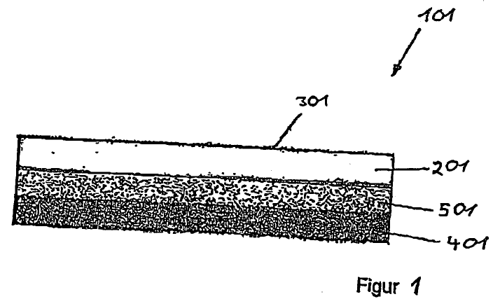

The invention is explained in the following by means of the Examples 1-2 and FIG. 1. These explanations are solely for example and do not restrict the general notions of the invention.

### EXAMPLE 1

Manufacture of the Ultraphobic Surface:

A roller-polished AlMg₃ sheet with a surface of 26 mm×76 mm and a thickness of 0.15 mm was degreased at room temperature with chloroform (p.a.) and afterwards 20 s in aqueous NaOH (5 g/l) at 50° C. Afterwards it was pickled in H₂PO₄ (100 g/l) for 20 s, rinsed in distilled water for 30 s and electrochemically pickled for 90 s in a mixture of HCl/H₃BO₃ (4 g/l each) at 35° C. and 120 mA/cm² at 35 V alternating current.

After 30 s rinsing in distilled water and 30 s alkaline rinsing in aqueous NaOH (5 g/l) it was rinsed again in distilled water for 30 s and anodically oxidised afterwards for 90 s in H₂SO₄ (200 g/l) at 25° C. with 30 mA/cm² at 50 V direct current.

Afterwards it was rinsed for 30 s in distilled water, then 60 s at 40° C. in NaHCO₃ (20 g/l), then again 30 s in distilled water and dried for 1 hour at 80° C. in the drying cabinet.

The sheet treated like this was coated with an approx. 40 nm thick layer of gold using cathodic evaporation in the high vacuum. Finally the sample was coated with a monolayer by immersion for 24 hours in a solution of the thiol CF₃—(CF₂)₇—(CH₂)₂—SH in benzotrifluoride (p.a. 1 g/l) at room temperature in a closed container, rinsed afterwards with benzotrifluoride (p.a.) and dried.

The surface shows a static contact angle of 178° for water. On inclining the surface by <2° a drop of water with a volume of 10 μl rolls away.

### EXAMPLE 2

A sample carrier with a surface according to Example 1 is used for this example. Various aliquots of MALDI matrices e.g. 3-hydroxypicolinic acid, sinapic acid and α-cyano-4-hydroxycoumarin acid dissolved in acetone, acetone nitrile or a mixture of water and one of the mentioned organic solvents, where the solvent content should be at least 50% by volume, were dispensed on the uncleaned ultraphobic surface with a piezo dispensing station. After the rapid evaporation of the solvent, all the tested matrices precipitated on the ultraphobic surface as hydrophilic areas in the form of small crystals and adhered so firmly to it that they could not be detached with either a wipe or with compressed air. The places populated with matrices had respectively a diameter from 200-1000 μm. The matrices are hydrophilic for the purpose of the invention; i.e. they show two functionalities. Various samples, respectively 0.5-2.0 μl which showed biomolecules were metered onto the places populated with matrices. The samples contained for example peptides or proteins dissolved in 0.1% TFA water or oligonucleotides dissolved in water where the content in the biomolecule amounted to 0.1-1 pmol per μl respectively. The samples were applied to the matrices with a hand pipette and evaporated at room temperature and analysed afterwards in a MALDI TOF mass spectrometer MTP Autoflex from the company Bruker Daltronic GmbH, 28359 Bremen in linear or reflector operation. In all cases, high-quality mass spectrums were recorded although the ultraphobic surface was not cleaned before each application. This is particularly important for analyses of nucleic acids which are falsified by the least amount of contamination for example by Na or K salts on the sample carriers.

FIG. 1 shows the membrane 101 which consists of a first layer 201 with an ultraphobic surface 301 and a carrier 401. The first layer 201 is fixed on the carrier using an adhesive layer 501. The expert realises that the adhesive layer 501 does not absolutely have to be present. The adhesive layer 501 consists of an electrically conductive material so that there is an electrical contact between the first layer 201 and the carrier.

1. Membrane with an ultraphobic surface and with at least one hydrophilic and/or oleophilic area, characterised in that the hydrophilic and/or oleophilic area shows at least one further functionality in addition to the affinity for water and/or oil.

2. Membrane according to claim 1 with a multitude of hydrophilic and/or oleophilic areas.

3. Membrane according to claim 1, characterised in that the hydrophilic and/or oleophilic areas are each completely enclosed by the ultraphobic surface.

4. Membrane according to claim 1, characterised in that the hydrophilic and/or oleophilic areas are distributed at least partly according to a defined pattern on the ultraphobic surface.

5. Membrane according to claim 1, characterised in that the ultraphobic surface shows a surface topography for which the local frequency f of the individual Fourier components and their amplitudes a(f) expressed by the integral S(log (f))=a(f)*f, calculated between the integration limits log(f₁ μm⁻¹)=3 and log (f₂ μm⁻¹)=3, is at least 0.3 and which consists of a hydrophobic and/or particularly durable oleophobic material.

6. Membrane according to claim 1, characterised in that the hydrophilic and/or oleophilic areas each show an area of 1 μm²-10 μm².

7. Membrane according to claim 1, characterised in that the hydrophilic areas are each at least one deposit, preferably a solidified substance, on the ultraphobic surface.

8. Membrane according to claim 1, characterised in that the hydrophilic areas are each a MALDI matrix.

9. Membrane according to claim 1, characterised in that the hydrophilic areas are each at least one affinity matrix.

10. Membrane according to claim 1, characterised in that the hydrophilic areas are each at least one substrate on which at least one molecule, especially a biomolecule, can be bonded.

11. Membrane according to claim 1, characterised in that the hydrophilic areas are each at least one biomolecule, preferably a DNA and/or a protein molecule.

12. Membrane according to claim 1, characterised in that the hydrophilic and/or oleophilic areas can be produced reversibly.

13. Membrane, preferably sample carrier, with several biomolecules which are each immobilised on an ultraphobic surface.

14. Membrane according to claim 13, characterised in that the biomolecules are arranged in the form of a specified raster.

15. Membrane according to claim 13, characterised in that the biomolecules are DNA and/or protein molecules.

16. Membrane, preferably sample carrier, with several MALDI matrices which are each immobilised on an ultraphobic surface.

17. Membrane according to claim 16, characterised in that the MALDI matrices are arranged in the form of a specified raster.

18. Membrane according to claim 16, characterised in that the MALDI matrices are 3-hydroxypicolinic acid, α-cyano-4-hydroxycoumarin acid, 2.5 dihydroxybenzoic acid, sinapic acid, 2,4,6 trihydroxyacetophenone or their mixture.

19. Membrane, preferably sample carrier, with several affinity matrices which are each immobilised on an ultraphobic surface.

20. Membrane according to claim 19, characterised in that the affinity matrices are arranged in the form of a specified raster.

21. Membrane according to claim 1 with a first layer with an ultraphobic surface and with a carrier, characterised in that the first layer is applied reversibly on a carrier and the maximum local flatness deviation is <100 μm.

22. Membrane according to claim 21, characterised in that the first layer is glued to the carrier.

23. Membrane according to claim 21, characterised in that there is an electrical contact between the first layer and the carrier.

24. Membrane according to claim 21, characterised in that the ultraphobic surface (3) shows at least one hydrophilic area.

25. Membrane according to claim 21, characterised in that the layer is disposable.

26. Process for maufacturing a membrane according to claim 1, characterised in that a functional substance, which is dissolved and/or suspended in a solvent that wets the ultraphobic surface is metered, preferably in drops, on to each ultraphobic surface and the liquid phase or the solvent is evaporated afterwards.

27. Process according to claim 21, characterised in that several different substances are metered on the ultraphobic surface.

28. Process according to claim 21, characterised in that the substances are MALDI matrices, affinity matrices, biomolecules, reagents and/or bond molecules.

29. Application of the membrane according to claim 1 in chemical research and in biotechnology.
